(19) United States
(12) Patent Application Publication
(10) Pub. No.: US 2016/0095861 A1
(43) Pub. Date: Apr. 7, 2016

(54) OPHTHALMIC OINTMENTS COMPRISING VALGANCICLOVIR HYDROCHLORIDE FOR TREATING INFECTIVE EYE DISEASES

(71) Applicant: SKIRON BIOSCIENCES, Albany, CA (US)

(72) Inventor: ANTON DELWIG, Albany, CA (US)

(73) Assignee: SKIRON BIOSCIENCES, Albany, CA (US)

(21) Appl. No.: 14/876,730

(22) Filed: Oct. 6, 2015

Related U.S. Application Data
(60) Provisional application No. 62/061,092, filed on Oct. 7, 2014.

(51) Int. Cl. A61K 31/522; A61K 9/00

(52) U.S. Cl. CPC A61K 31/522; A61K 9/0048

(57) ABSTRACT

The present invention provides ophthalmic ointments for treating infective eye diseases containing valganciclovir hydrochloride as an active ingredient in the amount of from about 0.01% to about 10.0% w/w. These ophthalmic ointments are particularly effective for treating eye diseases caused by herpesviruses in the eye. Compared with oral administration, topical administration of valganciclovir hydrochloride is not accompanied by systemic side effects such as bone marrow suppression. Compared with topical administration of ganciclovir, topical administration of valganciclovir hydrochloride is much more effective in treating infective diseases in the eye and is not accompanied by toxicity to the surface of the eye.

### CROSS-REFERENCES TO RELATED APPLICATIONS

The present application claims priority to U.S. Provisional Patent Application No. 62/061,092, filed on Oct. 7, 2014, which application is incorporated herein by reference in its entirety.

### BACKGROUND OF THE INVENTION

The present invention relates to ophthalmic ointments for treating infective eye diseases caused by herpesviruses, and more particularly, to ophthalmic ointments for treating infective eye diseases caused by cytomegalovirus (CMV).

Inflammation in the eye is one of the leading causes of blindness (Suttorp-Schulten et al., Br J Ophthalmol. 1996 September;80(9):844-8.). In recent years, CMV, a type of herpesviruses, has been found to be a common pathogen of inflammatory eye diseases such as Posner-Schlossman syndrome (PSS), Fuch's heterochromic iridocyclitis (FHI), corneal endotheliitis, hypertensive uveitis/iritis, glaucomatocyclitic crisis, inflammatory glaucoma or uveitic glaucoma (Alqahtani et al., Invest Ophthalmol Vis Sci. 2010;51:ARVO E-Abstract 2909). There is growing understanding that targeting CMV in patients with these eye diseases is an effective therapeutic strategy (Kim et al, Br J Ophthalmol. 2006 July; 90(7): 812-813.).

Ganciclovir is an anti-viral compound that has been approved for the treatment of CMV infections. Valganciclovir hydrochloride is the L-monovaline ester of ganciclovir and is a prodrug of ganciclovir with improved absorption. Valganciclovir hydrochloride is described in U.S. Pat. No. 6,083,953. Ganciclovir and valganciclovir are effective against all types of human herpesviruses, hepatitis B virus and several adenovirus strains (Colin, Clin Ophthalmol. 2007 December;1(4):441-53.). These viruses cause inflammation in all layers of the eye (cornea, conjuctiva, sclera, uvea and retina). In addition to the above-mentioned eye diseases associated with CMV infection, herpesviruses lead to eye diseases such as Herpes Zoster Ophthalmicus associated with varicella zoster virus (VZV) infection, herpetic keratitis (including dendritic corneal uclers, geographic corneal ulcers and stromal keratitis), uveitis and retinitis associated with herpes simplex virus (HSV) infection. Infections with adenoviruses lead to keratoconjunctivitis (including epidemic kerato-conjunctivitis (EKC) and pharyngoconjunctival fever). Hepatitis B virus (HBV) infections cause HBV-uveitis, retinitis and optic neuritis.

Valganciclovir hydrochloride is currently only available in solid dosage form for oral administration. Ganciclovir is available in various dosage forms including solid dosage form for oral administration, liquid dosage form for intravenous administration, solid dosage form for intraocular administration via intravitreous implant and as an ophthalmic gel for topical application in the eye.

Topical ophthalmic gel formulation of ganciclovir is approved for the treatment of herpetic diseases on the ocular surface (keratitis). However, it is not effective for the treatment of herpetic diseases inside the eye. This lack of efficacy is probably due to poor penetration through the cornea into the anterior chamber (Gunda et al., Journal Of Ocular Pharmacology And Therapeutics. 2006, 22(6): 465-476.). Ganciclovir, being an active compound and inhibitor of DNA replication, is also toxic to actively replicating corneal epithelial cells leading to epithelial erosion (punctate keratitis) if used for more than two weeks (Sahin et al., Ophthalmol Eye 2012 Dis. 4: 23-34.).

Systemic treatment with either oral valganciclovir or intravenous ganciclovir is very effective for the treatment of inflammation in the eye due to CMV infection (Chee et al., Br J Ophthalmol 2010;94:1648-1652.). However, systemic treatments have serious side effects, including bone marrow suppression and impairment of fertility (Valcyte, FDA Pharmacology Review, NDA 21-304).

Since CMV-induced inflammation in the eye is a chronic condition, in most cases requiring years of anti-viral therapy, there is need for a topical ophthalmic formulation of valganciclovir that is effective and non-toxic.

Accordingly, it is an objective of the present invention to provide an ophthalmic ointment for treating infective eye diseases, and in particular, to provide an ophthalmic ointment for treating infective eye diseases caused by CMV and other herpesviruses.

### BRIEF SUMMARY OF THE INVENTION

The present invention provides an ophthalmic ointment for treating infective eye diseases containing valganciclovir hydrochloride as an active ingredient in the amount of from about 0.01% to about 10.0% w/w (i.e., by weight of the total formulation).

In particular embodiments, the present invention is directed to an ophthalmic ointment containing valganciclovir hydrochloride as an active ingredient in the amount of from about 0.01% to about 10.0% w/w for treating infective eye diseases caused by cytomegalovirus (CMV), herpes simplex virus, other herpesviruses, hepatitis B virus, or adenoviruses. In preferred embodiments, the present invention is directed to an ophthalmic ointment for treating or preventing inflammation in the eye caused by CMV. Thus, in a more specific embodiment, the present invention provides an ophthalmic ointment for treating CMV infection in the eye containing as an active ingredient from about 0.01% to about 10.0% w/w of valganciclovir hydrochloride.

In some embodiments, the amount of valganciclovir hydrochloride in the ophthalmic ointment of the present invention ranges from about 0.01% to about 7.5% w/w, from about 0.01% to about 5.0% w/w, from about 0.01% to about 2.5% w/w, from about 0.1% to about 10.0% w/w, from about 0.1% to about 7.5% w/w, from about 0.1% to about 5.0% w/w, from about 0.1% to about 2.5% w/w, from about 0.5% to about 10.0% w/w, from about 0.5% to about 7.5% w/w, from about 0.5% to about 5.0% w/w, from about 0.5% to about 2.5% w/w, from about 1.0% to about 10.0% w/w, from about 1.0% to about 7.5% w/w, from about 1.0% to about 5.0% w/w, or from about 1.0% to about 2.5% w/w. In some embodiments, the amount of valganciclovir hydrochloride in the ophthalmic ointment of the present invention is about 0.01% w/w, 0.05% w/w, 0.1% w/w, 0.5% w/w, 1.0% w/w, 1.5% w/w, 2.0% w/w, 2.5% w/w, 3.0% w/w, 3.5% w/w, 4.0% w/w, 4.5% w/w, 5.0% w/w, 5.5% w/w, 6.0% w/w, 6.5% w/w, 7.0% w/w, 7.5% w/w, 8.0% w/w, 8.5% w/w, 9.0% w/w, 9.5% w/w, or 10% w/w. In particular embodiments, the amount of valganciclovir hydrochloride in the ophthalmic ointment of the present invention is within a range of from about 0.5% to about 5.0% w/w, e.g., about 1.0% w/w or about 2.0% w/w.

In certain embodiments, the ophthalmic ointment is an anhydrous formulation. In other embodiments, the ophthalmic ointment is an aqueous formulation.

Valganciclovir hydrochloride in oral dosage form is the agent of the first choice for treatment of CMV-induced inflammation in the eye. However, oral administration of valganciclovir is associated with serious side effects including bone marrow suppression and impairment of fertility. The first innovation of the present invention is that by topical application of valganciclovir in the form of an ophthalmic ointment, systemic toxicity can be avoided due to a much lower systemic exposure from a topical application to the eye. For example, topical application of 0.15% ganciclovir ophthalmic gel resulted in negligible amount of ganciclovir in blood (FDA Pharmacology Review, NDA 22-211).

Ganciclovir ophthalmic gel is the second choice for the treatment of CMV-induced inflammation in the eye. However, ganciclovir ophthalmic gel is not effective in most patients, probably due to poor penetration through the cornea into the anterior chamber. The second innovation of the present invention is that by using valganciclovir, which has much better permeability through the cornea into the anterior chamber, it is now possible to achieve a high enough concentration of the drug inside the eye to suppress CMV replication and to control CMV infection in the eye.

Additionally, the use of the existing ganciclovir ophthalmic gel is limited to two weeks because ganciclovir, being an active compound and inhibitor of DNA replication, is toxic to actively replicating corneal epithelial cells leading to epithelial erosions (punctate keratitis). CMV-induced inflammation in the eye, a chronic disease, requires years of treatment. Valganciclovir, a pro-drug of ganciclovir, is not active until it gets activated by esterases, which are located mostly inside the cornea. Therefore, the third innovation of the present invention is that by using valganciclovir, this toxicity to the surface of the eye can be avoided, thereby allowing chronic topical application to the eye for long periods of time.

Accordingly, the ophthalmic ointment of the present invention comprising valganciclovir hydrochloride overcomes the above-mentioned disadvantages and provides a highly effective therapeutic formulation for treating infective eye diseases such as CMV-induced inflammation in the eye.

No topical ophthalmic ointment containing valganciclovir hydrochloride has been proposed and no one has ever conceived of the idea of using valganciclovir hydrochloride in ophthalmic ointments. Considering the fact that no effective topical therapeutic formulation has ever been proposed in this field, the possible impact of the present invention will be of considerable medical importance. The present inventor prepared an ophthalmic ointment containing valganciclovir hydrochloride and discovered that the ointment exhibited excellent efficacy as a therapeutic formulation for treating infective eye diseases such as CMV-induced inflammation in the eye.

Other objects, features, and advantages of the present invention will be apparent to one of skill in the art from the following detailed description.

### BRIEF DESCRIPTION OF THE DRAWINGS

None

### DETAILED DESCRIPTION OF THE INVENTION

### I. Definitions

As used herein, the following terms have the meanings ascribed to them unless specified otherwise.

The term “infective eye disease” includes a clinical diagnosis that is associated with a herpesvirus, hepatits B virus, or adenovirus infection (e.g., inflammation) in the eye. Non-limiting examples of infective eye diseases associated with infection by a herpesvirus such as cytomegalovirus (CMV) include Posner-Schlossman syndrome (PSS), Fuch's heterochromic iridocyclitis (FHI), corneal endotheliitis, hypertensive uveitis/iritis, glaucomatocyclitic crisis, inflammatory glaucoma, uveitic glaucoma, and combinations thereof. Non-limiting examples of infective eye diseases associated with infection by other herpesviruses include Herpes Zoster Ophthalmicus (varicella zoster virus (VZV) infection), herpetic keratitis (including dendritic corneal uclers, geographic corneal ulcers, and stromal keratitis), uveitis, and retinitis (herpes simplex virus (HSV) infection), and combinations thereof. As a non-limiting example, infections with adenoviruses lead to keratoconjunctivitis (EKC) and/or pharyngoconjunctival fever. As another non-limiting example, Hepatitis B virus (HBV) infections cause HBV-uveitis, retinitis and/or optic neuritis.

“Inflammation” refers to white blood cell or leukocytic infiltration associated with infection and/or cellular injury.

“CMV-induced inflammation in the eye” includes, but is not limited to, inflammatory conditions of the palpebral and bulbar conjunctiva, cornea, eye lids, and anterior chamber of the eye (including corneal endothelium, trabecular meshwork, iris, and ciliary body) associated with the CMV infection.

The term “subject,” “patient,” or “individual” typically refers to humans, but also to other animals including, e.g., other primates, rodents, canines, felines, equines, ovines, porcines, and the like.

### II. Description of the Embodiments

In an ophthalmic ointment of the present invention containing valganciclovir hydrochloride as an active ingredient, the content (i.e., concentration) of valganciclovir hydrochloride is from about 0.01% to about 10.0% w/w, preferably from about 0.1% to about 5.0% w/w.

As described in the Examples below, the present inventor prepared and topically applied ophthalmic ointments of the invention containing valganciclovir hydrochloride at a concentration of 5.0% w/w or 1.0% w/w to an infected eye and discovered that the ointments were effective in controlling CMV-induced inflammation in the eye. In particular embodiments, the ophthalmic ointments of the invention are topically applied to a conjunctival sac of the eye.

A preferred ointment base used to prepare the ophthalmic ointment of the present invention may be one that has been used in conventional ophthalmic ointments. In particular, the preferred base may be mineral oil, white petrolatum, purified lanolin, hydrocarbon gel, polyethylene glycol, hydrophilic ointment base, white ointment base, absorptive ointment base, Macrogol (Trade Name) ointment base, simple ointment base, and mixtures thereof.

In particular embodiments, the ointment base is an anhydrous (i.e., non-aqueous) ointment base. In preferred embodiments, the anhydrous ointment base comprises or consists of mineral oil, white petrolatum, or mixtures thereof In other embodiments, the ointment base is an aqueous ointment base.

The ophthalmic ointment of the present invention may contain further conventional excipients other than the ointment base in the range of without affecting the intended functions and stability of valganciclovir hydrochloride to be contained. As non-limiting examples, the ophthalmic ointment may contain various excipients such as buffering agents (e.g., phosphate buffers, borate buffers, citrate buffers, tartarate buffers, acetate buffers, amino acids, sodium acetate, sodium citrate, and mixtures thereof), isotonicity agents (e.g., saccharides such as sorbitol, glucose and mannitol, polyhydric alcohols such as glycerin, concentrated glycerin, polyethylene glycol and propylene glycol, salts such as sodium chloride, and mixtures thereof), preservatives or antiseptics (e.g., benzalkonium chloride, benzethonium chloride, p-oxybenzoates such as methyl p-oxybenzoate or ethyl p-oxybenzoate, benzyl alcohol, phenethyl alcohol, sorbic acid or its salt, thimerosal, chlorobutanol, other quaternary amines, and mixtures thereof), solubilizing aids or stabilizing agents (e.g., cyclodextrins and their derivatives, water-soluble polymers such as polyvinyl pyrrolidone and carbomers, surfactants such as polysorbate 80 (Tween 80), polyoxyl 40 stearate, and polyoxyethylene hydrogenated castor oil, stabilizers such as sodium edetate and citric acid, salts thereof, and mixtures thereof), pH modifiers (e.g., hydrochloric acid, acetic acid, phosphoric acid, sodium hydroxide, potassium hydroxide, monium hydroxide, and mixtures thereof), thickening agents (e.g., hydroxyethyl cellulose, hydroxypropyl cellulose, methyl cellulose, hydroxypropylmethyl cellulose, carboxymethyl cellulose, salts thereof, and mixtures thereof), chelating agents (e.g., sodium edetate, sodium citrate, condensed sodium phosphate), and mixtures thereof. Descriptions of compounds used in standard ophthalmic formulations may be found in, for example, Remington's Pharmaceutical Sciences, latest edition, Mack Publishing Co. Easton Pa. Non-limiting examples of the contemplated excipients include a buffer, osmotic agent, demulcent, surfactant, stabilizers, emollient, tonicity agent, and/or a preservative component.

Non-limiting examples of additional excipients that can be used in the ophthalmic ointment of the invention include antiseptics such as parahydroxybenzoate, chlorobutanol, benzalkonium chloride, and mixtures thereof; surfactants such as polysorbate 80, polyoxyl 40 stearate, polyoxyethylene hydrogenated castor oil, and mixtures thereof; stabilizers such as sodium edetate, citric acid, salts thereof, and mixtures thereof; alcohols such as glycerol, lanolin alcohol, cetanol, and mixtures thereof; esters such as isopropyl myristate, ethyl linoleate, and mixtures thereof; and oils such as olive oil, triglycerides of middle-chained fatty acids, and mixtures thereof.

The ophthalmic ointment of the present invention can be produced as follows: if necessary, antiseptics, surfactants, stabilizers, alcohols, esters or oils are blended with an ointment base such as mineral oil or white petrolatum placed in a mortar or a mixing machine for ointment to form a mixture. This is followed by addition of valganciclovir hydrochloride, and the resulting mixture is mixed until uniform and kneaded to form the ophthalmic ointment. The ointment thus prepared is filled into a bottle or tube for ointment to obtain the ophthalmic ointment containing valganciclovir hydrochloride of the present invention.

The ophthalmic ointment containing valganciclovir hydrochloride of the present invention obtained in the above-described manner is efficacious against infective eye diseases caused by herpesviruses, hepatitis B virus, or adenoviruses, and is particularly effective for treating eye diseases associated with CMV infection including Posner-Schlossman syndrome (PSS), Fuch's heterochromic iridocyclitis (FHI), corneal endotheliitis, hypertensive uveitis/iritis, glaucomatocyclitic crisis, inflammatory glaucoma, and/or uveitic glaucoma.

### III. EXAMPLES

The present invention will now be described in detail with reference to specific examples, but it is to be noted that the present invention is not limited by these examples in any way.

### Example 1

### Preparation of 1% w/w of Valganciclovir Hydrochloride Ophthalmic Ointment

20 g of mineral oil and 79 g of white petrolatum were placed in a mortar and were mixed and kneaded until uniform. This was followed by addition of 1 g of valganciclovir hydrochloride and the resulting mixture was thoroughly kneaded to form a homogenous ophthalmic ointment containing 1% w/w of valganciclovir hydrochloride.

### Example 2

### Preparation of 5% w/w of Valganciclovir Hydrochloride Ophthalmic Ointment

40 g of mineral oil and 55 g of white petrolatum were placed in a mortar and were mixed and kneaded until uniform. This was followed by addition of 5 g of valganciclovir hydrochloride and the resulting mixture was thoroughly kneaded to form a homogenous ophthalmic ointment containing 5% w/w of valganciclovir hydrochloride.

### Example 3

### Preparation of 0.5% w/w of Valganciclovir Hydrochloride Ophthalmic Ointment

30 g of mineral oil and 69.5 g of white petrolatum were placed in a mortar and were mixed and kneaded until uniform. This was followed by addition of 0.5 g of valganciclovir hydrochloride and the resulting mixture was thoroughly kneaded to form a homogenous ophthalmic ointment containing 0.5% w/w of valganciclovir hydrochloride.

### Example 4

### Method of Treating CMV-Induced Inflammation in the Eye

The 1% w/w and 5% w/w valganciclovir hydrochloride ophthalmic ointments were tested in a patient with CMV-induced inflammation in the eye. After confirming the effectiveness of the oral valganciclovir hydrochloride (trade name Valcyte) to control the inflammation and to lower the intraocular pressure, the patient was switched to an ophthalmic ointment formulation of valganciclovir hydrochloride, which was applied once a day at night. Both 1% w/w and 5% w/w ointments were effective in controlling the disease. However, the 5% w/w ointment was associated with the appearance of punctate keritits and hyperemia. The 1% w/w ointment was equally effective in controlling the disease but was not associated with any side effects.

In view of the results described above, it can be concluded that the ophthalmic ointments of the present invention containing valganciclovir hydrochloride are capable of effectively controlling the inflammation and increased intraocular pressure associated with the CMV infection in the eye.

It should be noted that an ophthalmic ointment tends to remain in a conjunctival sac for a prolonged period of time while releasing drug in a sustained manner and the ophthalmic ointment of the present invention is considerably more effective as compared to an eye drop formulation of valganciclovir hydrochloride.

### Example 5

### Topical Valganciclovir for the Treatment of Hypertensive Anterior Uveitis

### Abstract

Purpose: To report the use of topical valganciclovir for the treatment of hypertensive anterior uveitis associated with clinical signs of cytomegalovirus (CMV) iritis.

Methods: A case report and review of the literature.

Results: A 37 year-old male was referred with a unilateral hypertensive anterior uveitis with keratic precipitates suggestive of CMV as the causative agent. After institution of oral valganciclovir and topical corticosteroids, the patient's ocular inflammation resolved and intraocular pressure normalized. Therapy was eventually changed from oral valganciclovir to ophthalmic 1% valganciclovir ointment, which was able to effectively control the ocular inflammation and allow the patient to discontinue topical corticosteroids and anti-hypertensive medications. Topical application of valganciclovir did not result in clinically evident ocular surface toxicity.

Conclusion: 1% valganciclovir ointment may prove to be an effective treatment of hypertensive anterior uveitis associated with clinical signs of CMV iritis.

### Introduction

Cytomegalovirus (CMV) has been recognized as a cause of hypertensive iritis in immunocompetent patients, including cases of Posner-Schlossman syndrome (PSS) and Fuchs' heterochromic iridocyclitis (FHI).¹ Clinical features of CMV iritis include stellate, circinate or coin shaped keratic precipitates (KPs), elevated intraocular pressure (TOP), and mild inflammation in the anterior chamber.¹⁻³ Current management of CMV iritis consists of appropriate antiviral medications along with topical steroids and topical anti-glaucoma agents as necessary. The main mode of antiviral therapy is oral valganciclovir, which shows very good efficacy.⁴ Additional approaches include intravenous ganciclovir, intravitreal ganciclovir, and topical ganciclovir.⁴ However, repeat injections of intravitreal ganciclovir has obvious limitations, as does the chronic use of intravenous ganciclovir or oral valganciclovir, whose long term side effects include bone marrow suppression and impairment of fertility.⁵ Although an ophthalmic gel formulation of ganciclovir (0.15%; Zirgan, Bausch & Lomb, Bridgewater, N J, USA), has been reported to control some patients with CMV iritis,⁴ this approach is not always successful. Since CMV anterior uveitis may require years of anti-viral therapy, there is need for a safe, effective and less expensive approach for managing CMV iritis. We report the successful treatment of a case of presumed CMV iritis by topical administration of valganciclovir.

### Formulation of 1% Topical Valganciclovir Ointment

Valganciclovir was extracted from the tablet form (450 mg, Valcyte, Roche). Briefly, tablets were dissolved in 0.001N HCl (2 ml per tablet), the insoluble excipients were isolated by centrifugation at 16,000 g, the solution was then filtered through 0.2 μm filter into ice-cold isopropanol (20 ml per tablet) leading to precipitation of the valganciclovir. The precipitate was spun down at 3,000 g, isopropanol was decanted and remaining volatile solvents were removed in the lyophilizer. The identity of valganciclovir was confirmed by liquid chromatography-mass spectrometry (LC-MS). 1% (w/w) valganciclovir ointment in the oleaginous base (20% mineral oil and 80% white petrolatum) was made by following standard compounding practices.

### Case Details

An otherwise healthy 28 year-old Caucasian male with a 2-year history of intermittent episodes of elevated intraocular pressure of the right eye was diagnosed with Posner-Schlossman syndrome. Management consisted of short courses of topical corticosteroids and anti-hypertensive medications with no systemic or topical antivirals. By his mid-30s, the frequency of attacks had increased to the point that chronic management was necessary. By this time, the patient had also developed iris atrophy, heterochromia and anisocoria.

The patient was referred to us at the age of 37 with chronic low-grade iritis and IOPs that were not adequately managed with medications (prednisolone acetate, brimonidine, and dorzolamide/timolol). Based on the history, pattern of keratic precipitates, mild anterior chamber inflammation, iris atrophy and elevated IOP (25 mmHg in the right eye and 16 mmHg in the left eye) CMV iritis was suspected. No anterior chamber reaction was noted. PCR of the aqueous was negative for CMV, herpes simplex virus (HSV), and varicella-zoster virus (VZV) DNA.⁶ However, because we had previously observed false negative PCR results in a substantial proportion of eyes suspected to have CMV iritis on clinical grounds, we decided to start a course of oral valganciclovir (900 mg twice daily). Within six weeks his IOP returned to normal levels and after 3 months all keratic precipitates completely resolved. Complete blood counts were tested periodically and no leukopenia developed. The dose of valganciclovir was then reduced to 450 mg twice daily and the disease remained quiet.

Nine months after the start of valganciclovir therapy, valganciclovir was discontinued and twice daily ganciclovir gel, 0.15%, was started. One month later, examination of the right eye revealed new keratic precipitates in a circinate pattern and slightly elevated intraocular pressure (23 mmHg). The patient refused to restart oral valganciclovir due to concerns about the toxicity of systemic valganciclovir, and continued with ganciclovir gel, topical corticosteroids, and topical anti-hypertensives. Within six months of discontinuing oral valganciclovir his IOP could not be controlled medically (topical anti-hypertensives and oral methazolamide), and a filtering procedure was scheduled.

At this same time, the patient began treating his condition with self-compounded topical valganciclovir. He initially wore a contact lens soaked overnight in 5% valganciclovir and later formulated 5% valganciclovir eye drops, which he administered 2-4 times per day. With this approach the patient's ocular inflammation and IOP improved and surgery was postponed, but the patient still required topical corticosteroids and topical anti-hypertensive medications. After 3 years of controlled inflammation on valganciclovir eye drops, the patient became concerned about medication stability and began formulating 1% valganciclovir ointment in an oleaginous base (80% white petrolatum and 20% mineral oil). Application of this ointment each night was effective in controlling the disease with no clinical evidence of ocular surface toxicity. With long-term use of topical valganciclovir ointment, the patient was able to taper off topical corticosteroids and topical anti-hypertensives and has remained stable since then (9 months).

### Discussion

This report demonstrates the successful use of topical valganciclovir for the treatment of hypertensive anterior uveitis. In our case, the decision to target CMV was based on the classic features of CMV iritis including coin shaped KP, elevated IOP and response to valganciclovir despite a single negative PCR-based assay for CMV DNA in the aqueous.

Currently, there is no safe and effective long-term maintenance therapy of CMV-associated anterior uveitis. Systemic therapy is effective but has serious potential side effects. Topical ganciclovir (0.15% gel; Zirgan) has been reported to be effective in a fraction of patients but has been ineffective in our experience, possibly due to poor corneal permeability.^{3,4,7} Besides the lack of efficacy, ocular surface toxicity is also a concern with long-term topical ganciclovir.

Topical valganciclovir may prove to be an option for long-term treatment of hypertensive iritis. Valganciclovir is about 8-fold more permeable through the cornea than ganciclovir.⁸ Valganciclovir may be less toxic to the corneal epithelium than ganciclovir because in order to become active valganciclovir needs to be hydrolyzed by esterases, which are located mostly inside the eye.⁹ Furthermore, a non-aqueous formulation of valganciclovir may be preferable to an aqueous formulation: first, because the non-aqueous base should prevent the hydrolysis of valganciclovir, which has a half-life of only 11 hours in water at neutral pH,¹⁰ and second, because a non-aqueous base may not require preservatives.

The morphology of the keratic precipitates was characteristic for CMV iritis, and the quick response to systemic valganciclovir was suggestive of CMV as the etiology. It is possible that a different herpes virus (e.g., HSV or VZV) was responsible for the hypertensive iritis since we did not attempt a course of acyclovir. However, even if that were the case, our findings are still noteworthy for suggesting that topical therapy with valganciclovir has a role in the treatment of hypertensive iritis.

In conclusion, this case report demonstrates that valganciclovir is effective in the treatment of an infective eye disease, and suggests that topical valganciclovir ointment could be an effective and safe long-term treatment for hypertensive anterior uveitis that is associated with clinical signs of CMV iritis.

### REFERENCES

1. Chee S P, Bacsal K, Jap A, et al. Clinical features of cytomegalovirus anterior uveitis in immunocompetent patients. Am J Ophthalmol. 2008;145:834-840.
2. Koizumi N, Suzuki T, Uno T, et al. Cytomegalovirus as an etiologic factor in corneal endotheliitis. Ophthalmology 2008;115:292-297.
3. Koizumi N, Inatomi T, Suzuki T, et al. Clinical features and management of cytomegalovirus corneal endotheliitis: analysis of 106 cases from the Japan corneal endotheliitis study. Br J Ophthalmol. 2015;99:54-58.
4. Chee S P, Jap A. Cytomegalovirus anterior uveitis: outcome of treatment. Br J Ophthalmol. 2010;94:1648-1652.
5. Segarra-Newnham M, Salazar M I. Valganciclovir: A new oral alternative for cytomegalovirus retinitis in human immunodeficiency virus-seropositive individuals. Pharmacotherapy. 2002;22:1124-1128.
6. Cohen J I, Fahle G, Kemp M A, Apakupakul K, Margolis T P. Human herpesvirus 6-A, 6-B, and 7 in vitreous fluid samples. J Med Virol. 2010;82:996-999.
7. Gunda S, Hariharan S, Mitra A K. Corneal absorption and anterior chamber pharmacokinetics of dipeptide monoester prodrugs of ganciclovir (GCV): in vivo comparative evaluation of these prodrugs with Val-GCV and GCV in rabbits. J Ocul Pharmacol Ther. 2006;22:465-476.
8. Majumdar S, Nashed Y E, Patel K, et al. Dipeptide monoester ganciclovir prodrugs for treating HSV-1-induced corneal epithelial and stromal keratitis: in vitro and in vivo evaluations. J Ocul Pharmacol Ther. 2005;21:463-474.
9. Lee V H. Esterase activities in adult rabbit eyes. J Pharm Sci. 1983;72:239-244.
10. Stefanidis D, Brandl M. Reactivity of valganciclovir in aqueous solution. Drug Dev Ind Pharm. 2005;31:879-884.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

What is claimed is:

1. An ophthalmic ointment for treating an infective eye disease comprising as an active ingredient from about 0.01% to about 10.0% w/w of valganciclovir hydrochloride.

2. The ophthalmic ointment of claim 1, wherein the eye disease is caused by a herpesvirus, hepatitis B virus, or an adenovirus.

3. The ophthalmic ointment of claim 2, wherein the herpesvirus is a cytomegalovirus (CMV), varicella zoster virus (VZV), or herpes simplex virus (HSV).

4. The ophthalmic ointment of claim 3, wherein the infective eye disease caused by CMV is selected from the group consisting of Posner-Schlossman syndrome (PSS), Fuch's heterochromic iridocyclitis (FHI), corneal endotheliitis, hypertensive uveitis/iritis, glaucomatocyclitic crisis, inflammatory glaucoma, uveitic glaucoma, and combinations thereof.

5. The ophthalmic ointment of claim 3, wherein the infective eye disease caused by VZV is herpes zoster ophthalmicus.

6. The ophthalmic ointment of claim 3, wherein the infective eye disease caused by HSV is selected from the group consisting of herpetic keratitis, dendritic corneal uclers, geographic corneal ulcers, stromal keratitis, and combinations thereof.

7. The ophthalmic ointment of claim 2, wherein the infective eye disease caused by hepatitis B virus is selected from the group consisting of HBV-uveitis, retinitis, optic neuritis, and combinations thereof.

8. The ophthalmic ointment of claim 2, wherein the infective eye disease caused by an adenovirus is selected from the group consisting of keratoconjunctivitis (EKC), pharyngoconjunctival fever, and combinations thereof.

9. The ophthalmic ointment of claim 1, wherein the infective eye disease is inflammation in any of the layers of the eye.

10. The ophthalmic ointment of claim 1, wherein the valganciclovir hydrochloride is present in an amount of from about 0.1% to about 5.0% w/w.

11. The ophthalmic ointment of claim 1, wherein the valganciclovir hydrochloride is present in an amount of about 1.0% w/w.

12. The ophthalmic ointment of claim 1, wherein the valganciclovir hydrochloride is present in an amount of about 2.0% w/w.

13. The ophthalmic ointment of claim 1, further comprising an ointment base.

14. The ophthalmic ointment of claim 13, wherein the ointment base is selected from the group consisting of mineral oil, white petrolatum, purified lanolin, hydrocarbon gel, a polyethylene glycol, hydrophilic ointment base, white ointment base, simple ointment base, and mixtures thereof.

15. The ophthalmic ointment of claim 13, wherein the ointment base is anhydrous.

16. The ophthalmic ointment of claim 15, wherein the anhydrous ointment base is mineral oil, white petrolatum, or a mixture thereof.

17. The ophthalmic ointment of claim 13, further comprising an excipient selected from the group consisting of antiseptics, surfactants, stabilizers, alcohols, esters, oils, and mixtures thereof.

18. The ophthalmic ointment of claim 1, wherein the ophthalmic ointment is an anhydrous formulation.

19. A method for treating an infective eye disease in a subject comprising topically administering an effective amount of the ophthalmic ointment of claim 1 to the subject.

20. The method of claim 19, wherein the subject is a human suffering from the infective eye disease.
